SEQUENCE LISTING (5 sequences)

SEQ ID NO: 1 - protein, 258 aa, artificial sequence
  LC-Cutinase
SNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 2 - protein, 258 aa, artificial sequence
  Poly(ethylene terephthalate) hydrolase [bacterium HR29]
SNPYQRGPNPTRSALTTDGPFSVATYSVSRLSVSGFGGGVIYYPTGTTLTFGGIAMSPGYTADASSLAWLGRRLASHGFVVIVINTNSRLDFPDSRASQLSAALNYLRTSSPSAVRARLDANRLAVAGHSMGGGATLRISEQIPTLKAGVPLTPWHTDKTFNTPVPQLIVGAEADTVAPVSQHAIPFYQNLPSTTPKVYVELDNATHFAPNSPNAAISVYTISWMKLWVDNDTRYRQFLCNVNDPALSDFRSNNRHCQ

SEQ ID NO: 3 - protein, 259 aa, artificial sequence
  enzyme
MANPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELRGASHLVSNTPDTTTAKYSIAWLKRFVDDDLRYEQFLCPAPDDFAISEYRSTCPF

SEQ ID NO: 4 - protein, 263 aa, artificial sequence
  enzyme Ideonella sp.
QTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 5 - protein, 603 aa, artificial sequence
  MHET hydrolase Ideonella sp.
MQTTVTTMLLASVALAACAGGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

(19) United States
(12) Patent Application Publication
(10) Pub. No.: US 2022/0002516 A1
(43) Pub. Date: Jan. 6, 2022

(54) METHOD FOR PRODUCING TEREPHTHALIC ACID ON AN INDUSTRIAL SCALE

(71) Applicant: CARBIOS, SAINT-BEAUZIRE (FR)

(72) Inventor: MICHEL CHATEAU, RIOM (FR)

(21) Appl. No.: 17/291,290

(22) PCT Filed: Nov. 5, 2019
(86) PCT No.: PCT/EP2019/080277

§ 371 Date: May 5, 2021

(30) Foreign Application Priority Data
Nov. 6, 2018 (FR) 1860220

(51) Int. Cl. C08J 11/10

(52) U.S. Cl. CPC C08J 11/105; C08J 2367/02

(57) ABSTRACT

The present invention relates to a process for producing terephthalic acid on an industrial or semi-industrial scale by enzymatic means from a polyester of interest.

### TECHNICAL FIELD OF THE INVENTION

The present invention relates to a process for producing terephthalic acid on an industrial or semi-industrial scale by enzymatic means from a polyester of interest.

### BACKGROUND ART

Plastics products are durable, inexpensive materials that can be used to manufacture a wide variety of products for various applications (food packaging, clothing textiles, etc.). Consequently, the production of plastics has dramatically increased in recent decades. Most are used for short-term applications, which results in an accumulation of plastic waste and a need for its treatment. The different polymers that make up these plastics include polyethylene terephthalate (PET), an aromatic polyester produced from terephthalic acid and ethylene glycol, which is used in many applications such as food packaging (bottles, flasks, jars, trays, pouches), but also in the production of textiles for clothing, decoration (carpeting), household linen, etc.

In order to address the environmental and economic problems of waste accumulation, recycling or energy recovery technologies have been developed. The mechanical recycling process remains the most commonly used today, but it has many drawbacks. Indeed, it requires sophisticated and costly sorting to implement and leads to the production of recycled plastics of diminished quality intended for applications of lesser value (lower molecular weight, uncontrolled presence of additives). Moreover, these recycled plastics are not competitive with virgin plastics derived from oil.

Recently, innovative processes for enzymatic recycling of plastic products have been developed and described in particular in patent applications WO 2014/079844, WO 2015/097104, WO 2015/173265 and WO 2017/198786. Unlike conventional mechanical recycling processes, these enzymatic processes allow, by enzymatic depolymerization of the polymer contained in the plastic, to return to the main constituents (monomers) of the polymer. The monomers obtained can then be purified and used to repolymerize new polymers. These enzymatic processes make it possible, via the specificity of the enzymes, to avoid a costly sorting of plastics, but also to propose an infinite recycling leading to recycled polymers of equivalent quality to the polymers derived from oil. In particular, these processes make it possible to produce terephthalic acid and ethylene glycol from PET.

One of the problems associated with the production of monomers derived from depolymerization is the step of recovering said monomers. Indeed, it is difficult to separate the monomers in solid form, such as terephthalic acid, from the rest of the solid waste present in the reactor, and in particular from the polyester not yet depolymerized. Such a recovery step is complex, costly, and makes it poorly compatible with industrial-scale use.

By working on these issues, the Applicants have developed an optimized enzymatic process, allowing the industrial-scale production of terephthalic acid from plastics and/or textiles containing a polyester comprising terephthalic acid, and in particular PET.

### SUMMARY OF THE INVENTION

The inventor has developed a process for producing terephthalic acid from at least one polyester comprising terephthalic acid, leading to a high-concentration production of terephthalic acid, thereby addressing the technical and economic constraints of industrial-scale production. More precisely, the inventor has developed a process for introducing a high concentration of polyester into a reactor while maintaining a depolymerization rate of said polyester compatible with industrial viability. Particularly, the inventor has identified that regulating the pH between 6.5 and 9 in a reactor, under stirring, allows a significant portion of the terephthalic acid produced to be maintained in soluble form. The high concentration of soluble terephthalic acid is particularly advantageous, as it simplifies the recovery step of these monomers and thus reduces production costs.

Moreover, the process developed by the inventor makes it possible to maintain depolymerization rates inside the reactor that are compatible with industrial-scale implementation. By way of example, the inventor succeeded in depolymerizing more than 90% of a polyester of interest containing terephthalic acid in only 24 h, resulting in the recovery of more than 90% of the terephthalic acid present in the polyester of interest. The process which is the object of the invention can be carried out on any plastic waste containing a polyester comprising terephthalic acid. The plastic waste can be fed directly into the reactor, without sophisticated sorting or elaborate pretreatment. Advantageously, the process of the invention can be implemented for the depolymerization and/or recycling of plastics. The process of the invention can be implemented for the recycling of polyesters comprising at least one terephthalic acid unit, primarily for the recycling of semi-aromatic polyesters, in particular selected from polyethylene terephthalate (PET), polyethylene terephthalate glycol (PETG), polyethylene co-isosorbide-terephthalate (PEIT), polytrimethylene terephthalate (PTT), polybutylene adipate terephthalate (PBAT), polycyclohexylenedimethylene terephthalate (PCT) and polybutylene terephthalate (PBT).

The invention therefore has as its object a process for producing terephthalic acid (TA) from at least one polyester of interest comprising at least one TA unit, comprising a step of enzymatic depolymerization of the polyester according to which said polyester is brought into contact with at least one enzyme capable of depolymerizing said polyester in a stirred reactor, and a step of recovering TA salts in solubilized form, characterized in that the amount of polyester introduced into the reactor is greater than 10% by weight based on the total weight of the initial reaction medium, in that the pH is regulated between 6.5 and 9 during the depolymerization step, and in that the concentration of TA in the liquid phase of the final reaction medium is greater than 40 kg/t.

Preferentially, the step of recovering the solubilized TA salts comprises a step of separating the liquid phase containing the TA salts from the rest of the final reaction medium.

Preferably, the polyester of interest is selected from PTT, PBAT, PBT, PET, PETG, PEIT, PCT. More preferentially the polyester of interest is PET.

Advantageously, the polyester of interest is introduced into the reactor in the form of powder and/or granules, in particular in the form of powder and/or granules with a particle size of less than 2 mm, preferentially less than 1 mm.

Advantageously, the depolymerization step of the process of the invention lasts at most 150 h, and more preferentially at most 48 h. Furthermore, the process according to the invention can be implemented in industrial-sized reactors, and in particular reactors having a useful volume of several liters, several tens of liters, several hundreds of liters.

Preferentially, the pH is regulated during the depolymerization step by the addition to the reaction medium of a basic solution concentrated to at least 10%±1%.

The invention also has as its object a process for recycling a polyester of interest comprising at least one TA unit, more particularly PET, comprising a step of enzymatic depolymerization of the polyester by bringing said polyester of interest into contact with at least one enzyme capable of depolymerizing said polyester, said depolymerization step being carried out in a stirred reactor, according to which the reactor contains an amount of engaged polyester greater than 10% by weight based on the total weight of the initial reaction medium, the pH being regulated between 6.5 and 9 during the depolymerization step, and a step of recovering the terephthalic acid salts in solubilized form.

The invention also has as its object a process for recycling a polyester of interest comprising at least one TA unit, comprising a step of enzymatic depolymerization of the polyester by bringing said polyester of interest into contact with at least one enzyme capable of depolymerizing said polyester, said depolymerization step being carried out in a reactor under stirring, according to which the reactor contains an amount of engaged polyester comprised between 15% and 25% by weight based on the total weight of the initial reaction medium, the pH is regulated between 7.5 and 8.5 during the depolymerization step by the addition to the reaction medium of a basic solution concentrated to at least 15%±1%, and in that the concentration of TA in the liquid phase of the final reaction medium is greater than 100 kg/t.

Advantageously, the recovered TA salts can be reused in the form of TA, in particular for the production of new polyesters.

Another object of the invention is the use of a reactor with a volume greater than 1 L, preferentially greater than 10 L, 100 L, 1000 L, for the implementation of the above-described processes.

### DETAILED DESCRIPTION OF THE INVENTION

### Definitions

In the context of the invention the expression “plastic material” refers to plastic products (such as sheets, trays, films, tubes, blocks, fibers, fabrics, etc.) and to the plastic compositions used to make the plastic products. Preferentially, the plastic material is composed of amorphous and/or semi-crystalline polymers. The plastic material may contain, in addition to the polymer(s), additional substances or additives, such as plasticizers, mineral or organic fillers, dyes, etc. Thus, in the context of the invention, plastic material refers to any plastic product and/or plastic composition comprising at least one polymer in semi-crystalline and/or amorphous form, more particularly at least one polyester.

Plastic products refer to manufactured plastic products, such as rigid or flexible packaging (films, bottles, trays), agricultural films, bags, disposable objects, textiles, fabrics, non-wovens, floor coverings, plastic waste or fiber waste, etc.

The term “polymer” refers to a chemical compound whose structure consists of multiple repeating units (i.e., “monomers”) linked by chemical covalent bonds. In the context of the invention, the term “polymer” refers more specifically to such chemical compounds used in the composition of plastic materials.

The term “polyester” refers to a polymer that contains an ester functional group in the main chain of its structure. The ester functional group is characterized by a bond between a carbon and three other atoms: a single bond with another carbon atom, a double bond with an oxygen and a single bond with another oxygen atom. The oxygen bonded to the carbon by a single bond is itself bonded to another carbon by a single bond. Polyesters can be made of only one type of monomer (i.e., homopolymer) or of at least two different monomers (i.e., copolymer). The polyesters can be aromatic, aliphatic or semi-aromatic. By way of example, polyethylene terephthalate is a semi-aromatic copolymer composed of two monomers, terephthalic acid and ethylene glycol. In the context of the invention, “polyester of interest” refers to a polyester comprising at least one terephthalic acid unit as a monomer.

In the context of the invention, the term “semi-crystalline polymers” refers to partially crystalline polymers, in which crystalline and amorphous regions coexist. The degree of crystallinity of a semi-crystalline polymer can be estimated by various analytical methods and is generally comprised between 10% and 90%. A polymer with a degree of crystallinity of less than 10% can be considered amorphous. In the present application, crystallinity is measured by differential scanning calorimetry (DSC). X-ray diffraction can also be used to measure the degree of crystallinity.

The term “depolymerization”, in relation to a polymer or to a plastic material containing a polymer, refers to a process by which a polymer or at least one polymer of said plastic material is depolymerized into smaller molecules, such as monomers and/or oligomers.

As used in the present application, the terms “solubilized” or “in solubilized form” refer to a compound dissolved in a liquid, as opposed to undissolved solid forms.

The term “terephthalic acid” or “TA” refers to the terephthalic acid molecule alone, i.e., C₈H₆O₄, corresponding to terephthalic acid in its acid form. The terms “terephthalic acid salts”, “terephthalate salts” or “TA salts” refer to a compound comprising a terephthalic acid molecule associated with a cation(s) such as sodium, potassium, ammonium. In the context of the invention, TA salts may include terephthalate disodium C₈H₄Na₂O₄, terephthalate dipotassium C₈H₄K₂O₄, terephthalate diammonium C₈H₁₂N₂O₄, terephthalate monosodium C₈H₅NaO₄, terephthalate monopotassium C₈H₅KO₄ and/or terephthalate monoammonium C₈H₁₀NO₄.

According to the invention, the concentration of terephthalic acid in the liquid phase of the final reaction medium corresponds to the amount of TA measured at the conclusion of the depolymerization step, regardless of its form, i.e., TA in solubilized or non-solubilized form, including in salt form. The concentration of terephthalic acid can be determined by any means known to the person skilled in the art, in particular by HPLC.

In the context of the invention, “engaged amount” refers to the amount of a compound, for example the amount of polyester of interest or the amount of enzyme, fed into the reactor at the beginning (time t=0) of the polyester depolymerization step. The engaged amount of polyester, and in particular of PET, refers to the amount of that polyester, independent of other compounds that may be present in the plastic material. Thus, in the case where the polyester is contained in a plastic waste, the engaged amount of said polyester is different from the engaged amount of plastic waste, as said plastic waste may contain other compounds in addition to said polyester.

“Reaction medium” means all the material (including in particular liquid, enzymes, the polyester of interest and/or the monomers resulting from the depolymerization of said polyester) present in the reactor during the depolymerization step, i.e., the contents of the reactor. “Initial reaction medium” and “final reaction medium” mean, respectively, the reaction medium at the beginning and at the conclusion of the depolymerization step. In the context of the invention, the total volume of the reactor is advantageously at least 10% greater than the volume of the final reaction medium.

“Liquid phase of the final reaction medium” means the reaction medium obtained at the conclusion of the depolymerization step, free of solid and/or suspended particles. Said liquid phase includes the liquid and all the compounds dissolved in this liquid (including enzymes, monomers, salts, etc.). This liquid phase can be obtained by separation from the solid phase of the reaction medium, using conventional techniques known to the person skilled in the art, such as filtration, centrifugation, etc. In the context of the invention, the liquid phase is in particular free of residual polyester, i.e., not degraded at the conclusion of the depolymerization step.

### Depolymerization Process

The process for producing terephthalic acid according to the invention is based on enzymatic depolymerization of at least one polyester of interest containing in its constituents at least one terephthalic acid unit, by contacting said polyester of interest with at least one enzyme capable of depolymerizing said polyester. More particularly, the inventor has developed a process for producing large amounts of terephthalic acid in an easily purifiable form, in a relatively short reaction time. Indeed, the inventor has unexpectedly discovered that it is possible to feed large loads of polyester of interest and at least one enzyme capable of depolymerizing it into a reactor, under stirring and maintaining a pH between 6.5 and 9, and to obtain a particularly high depolymerization rate resulting in terephthalic acid concentrations of more than 40 kg/t in the liquid phase of the reaction medium in a time which is perfectly acceptable on an industrial and semi-industrial scale. The process according to the invention also makes it possible to obtain terephthalic acid in solubilized form, i.e., in the form of TA salts, which allows it to be purified easily, making the process according to the invention particularly advantageous on an industrial scale.

The process for producing terephthalic acid (TA) according to the invention thus comprises
a step of depolymerizing the polyester of interest according to which said polyester is brought into contact with at least one enzyme capable of depolymerizing said polyester in a reactor under stirring, and
a step of recovering TA salts in solubilized form,

characterized in that the reactor contains, at the beginning of the depolymerization step, an amount of engaged polyester greater than 10% by weight based on the total weight of the initial reaction medium, in that the pH is regulated between 6.5 and 9 during the depolymerization step, and in that the concentration of TA in the reactor at the conclusion of the depolymerization step is greater than 40 kg/t in the liquid phase of the final reaction medium.

Advantageously, the typology of the terephthalic acid salts obtained is related to the base used to regulate the pH. Preferentially, the terephthalate salts produced during the depolymerization step are in the form of sodium terephthalate, potassium terephthalate and/or ammonium terephthalate.

According to the invention, with a regulated pH greater than or equal to 6.5, at the conclusion of the depolymerization step, TA salts are recovered in solubilized form in the liquid phase of the reaction medium.

The process according to the invention can be implemented in a reactor with a volume greater than 500 milliliters (mL), 1 liter (L), preferentially greater than 2 L, 5 L, 10 L. In a particular embodiment, the process of the invention can be implemented on an industrial and semi-industrial scale. Thus, it is possible to use a reactor whose volume is greater than 100 L, 150 L, 1000 L, 10 000 L, 100 000 L, 400 000 L. Preferentially, the process is implemented in a reactor with a volume greater than 1000 L.

### Reactor Contents

The initial reaction medium in the reactor comprises at least the polyester of interest, optionally contained in a plastic material and in particular in a plastic product or a plastic waste, the enzyme degrading said polyester and a liquid. As the depolymerization step proceeds, the reaction medium is enriched in monomers and in particular in TA salts and the amount of polyester of interest decreases.

Preferably, the liquid in the reactor comprises an aqueous solvent, preferentially water. In a preferred case, the liquid in the reactor is free of non-aqueous solvent, and in particular free of organic solvent. In an embodiment, the liquid in the reactor comprises only water.

According to the invention, the polyester of interest comprises at least one terephthalic acid unit as a monomer. Advantageously, the polyester of interest is selected from polytrimethylene terephthalate (PTT), polybutylene adipate terephthalate (PBAT), polybutylene terephthalate (PBT), polyethylene terephthalate (PET), poly(ethylene co-isosorbide-terephthalate) PEIT, polycyclohexylenedimethylene terephthalate (PCT), and/or copolymers of these. Preferentially, the polyester of interest is PET. In a particular case, the polyester of interest is selected from modified polyesters, preferentially the polyester of interest is modified PET, such as PET glycol (PETG).

Advantageously, the engaged amount of polyester of interest in the reactor is greater than or equal to 11% by weight based on the total weight of the initial reaction medium, preferentially greater than or equal to 15%, preferentially greater than or equal to 20%. Particularly, the engaged amount of polyester of interest in the reactor is less than 60% by weight based on the total weight of the initial reaction medium, preferentially less than 50%. In another particular case, the engaged amount of polyester in the reactor is comprised between 15% and 25% by weight based on the total weight of the initial reaction medium, preferentially 20%±2%. In another particular case the engaged amount of polyester in the reactor is comprised between 11% and 20% by weight based on the total weight of the initial reaction medium, preferentially 15%±2%. In an embodiment, the engaged amount of polyester of interest in the reactor is comprised between 11% and 60% by weight based on the total weight of the initial reaction medium, preferentially between 15% and 50%, more preferentially between 15% and 40%, between 15% and 30%, between 15% and 25%, between 20% and 30%, between 20% and 25%. In the case where several polyesters containing at least one terephthalic acid unit are used in the reactor, the amount of polyester used refers to the cumulative amounts of each of the polyesters.

According to the invention, the polyester of interest is an amorphous and/or semi-crystalline polyester. Preferably, the polyester of interest has a degree of crystallinity of less than 30%, preferentially less than 25%, more preferentially less than 20%. Particularly the polyester of interest has a degree of crystallinity less than 30%±10%, preferentially less than 25%±10%, more preferentially less than 20%±10%. In another preferred case, the polyester of interest is an amorphous polyester. According to the invention, it is possible to carry out a step of amorphization of the polyester of interest before the depolymerization step by any means known to the person skilled in the art. Such an amorphization step is described in particular in the application WO 2017/198786. In a particular embodiment, the polyester of interest or the plastic material containing the polyester of interest engaged in the reactor is in the form of granules or microgranules of a size of less than 5 mm, preferentially of a particle size of less than 3 mm, more preferentially of a particle size of less than 2 mm. According to the invention, it is possible to carry out a step of pre-treatment of the polyester of interest, and in particular a step of grinding the polyester of interest, or the plastic material containing the polyester of interest before the polyester depolymerization step. In a preferred embodiment, the polyester of interest or the plastic material containing the polyester of interest is reduced to powder form by any suitable means known to the skilled person. In this particular case, the polyester of interest, or the plastic material containing the polyester of interest, is advantageously micronized, so as to be converted into powder form.

In a particular embodiment, the production process comprises a step of amorphization of the polyester of interest, followed by a step of grinding and/or micronization of the polyester of interest or the plastic material containing the polyester of interest prior to the polyester depolymerization step.

In a particular embodiment, the polyester of interest or the plastic material containing the polyester of interest engaged in the reactor is in the form of powder and/or granules with an average particle size (d50) of less than 2 mm, preferentially with a particle size of less than 1 mm. In another embodiment, the polyester of interest or the plastic material containing the polyester of interest engaged in the reactor is in the form of a powder with an average particle size (d50) of less than 500 μm.

Preferentially, the polyester of interest has a degree of crystallinity of less than 25%±10%, and is engaged in the reactor in the form of powder and/or granules of a size less than 2 mm, preferentially less than 1 mm. According to the invention, it is possible to load the reactor directly with the polyester of interest, or with plastic materials containing at least the polyester of interest.

According to the invention, the plastic material(s) engaged in the reactor may contain a mixture of several polymers and in particular several polyesters. In a particular embodiment, the depolymerization process according to the invention is carried out with a plastic material comprising at least PET. In a preferred embodiment, PET represents at least 80% by weight based on the total weight of said plastic material, preferentially at least 85%, 90%, 95%. In a particular embodiment, the plastic material comprises a mixture of PET and polylactic acid (PLA), a mixture of PET and polyethylene (PE), a mixture of PET and polytrimethylene terephthalate (PTT), a mixture of PET and polyamide (PA), or a mixture of PET and cotton. Advantageously, the plastic materials used in the reactor are plastic waste or fiber waste. These waste materials may come from the collection channels intended for recycling, but may also be waste materials from the production channel or the recycling channel, and may thus contain compounds other than waste plastics. This implies that the polyester of interest can be engaged in the reactor in combination with other elements present in these flows (such as paper, cardboard, aluminum, glue, etc.). In a particular embodiment, the reactor is loaded with several plastic materials containing at least the polyester of interest, preferentially at least PET, more preferentially containing at least 80% PET. In another particular embodiment, the plastic material is selected from fibers and/or fiber and/or textile wastes and PET represents at least 60% by weight based on the total weight of said plastic material, preferentially at least 65%, 70%, 75%, 80%, 85%, 90%, 95%.

Advantageously, the enzyme degrading the polyester of interest is selected from cutinases, lipases and esterases degrading said polyester. In particular, said enzyme is selected from esterases degrading said polyester of interest. In a particular embodiment, said polyester is PET and the enzyme is a PET-degrading cutinase. More particularly, the enzyme is a cutinase preferentially from Thermobifida cellulosityca, Thermobifida halotolerans, Thermobifida fusca, Thermobifida alba, Bacillus subtilis, Fusarium solani pisi, Humicola insolens (such as that under entry A0A075B5G4 in the UniProt database), Sirococcus conigenus, Pseudomonas mendocina, and Thielavia terrestris, or a variant thereof. In another case, the cutinase is selected from cutinases from metagenomic libraries such as LC-Cutinase described in Sulaiman et al., 2012 or variants thereof. In another case, the enzyme is a lipase, preferentially from Ideonella sakaiensis. In an alternative case, the enzyme is selected from commercial enzymes such as Novozym 51032 or variants thereof. Of course, it is possible to load the reactor with several enzymes, and in particular at least two enzymes among those mentioned above.

In a particular case, the enzyme (or enzymes) is selected from enzymes having an amino acid sequence having at least 75% identity with SEQ ID NO: 1 and/or with SEQ ID NO: 2 and/or with SEQ ID NO: 3 and/or with SEQ ID NO: 4 and/or with SEQ ID NO: 5, and having activity of depolymerizing a polyester comprising at least one terephthalic acid unit. In a particular case, the enzyme is selected from enzymes having an amino acid sequence having at least 75% identity with SEQ ID NO: 1, and PET depolymerizing activity.

In a particular embodiment the enzyme is able to depolymerize the polymer to oligomers, in which case it is advantageously associated with an enzyme able to depolymerize said oligomers to monomers. In a particular example, the two enzymes are thus selected from the enzymes having an amino acid sequence having at least 75% identity with SEQ ID NO: 4 and/or SEQ ID NO: 5.

The inventor has identified that the process of the invention is particularly suitable in the particular case where the selected enzyme has an amino acid sequence having at least 90% identity with SEQ ID NO: 1 and the polyester of interest is preferentially selected from PET and/or PBAT. This is particularly the case with enzymes having the amino acid sequence of SEQ ID NO: 1 or SEQ ID NO: 2. Unlike other enzymes known to depolymerize polyesters, these enzymes experience limited inhibition of their activity by the monomers produced under the conditions of the process of the invention.

It is therefore an object of the invention to propose a process for producing terephthalic acid as described above and characterized in that the enzyme capable of depolymerizing said polyester is selected from enzymes having an amino acid sequence having at least 90% identity with SEQ ID NO: 1 and an activity of depolymerizing a polyester comprising at least one terephthalic acid unit and more particularly a PET depolymerizing activity.

Preferentially, the process for producing terephthalic acid according to the invention is carried out using PET and at least one enzyme capable of depolymerizing said PET selected from cutinases, as described above.

According to the invention, the amount of enzyme degrading the polyester of interest engaged in the reactor is advantageously sufficient to allow total or quasi-total depolymerization of said polyester (i.e., up to at least 80% by weight based on the weight of said engaged polyester) in reaction times compatible with industrial-scale implementation. In an embodiment, the ratio by weight of the amount of engaged enzyme to the amount of engaged polyester is comprised between 0.01:1000 and 3:1000. Preferentially the ratio of the amount of engaged enzyme to the amount of engaged polyester is comprised between 0.5:1000 and 2.5:1000, more preferentially between 1:1000 and 2:1000. In a particular case, the amount of engaged enzyme is greater than or equal to the amount of enzyme required to reach a saturating enzyme concentration, i.e., a concentration above which the reaction rate is not improved by the addition of enzyme. In a particular case, the enzyme may be engaged in the form of a composition comprising in addition to the enzyme excipients, which may be selected from buffers commonly used in biochemistry, preservatives, and/or stabilizing agents. The amount of enzyme then advantageously refers to the amount of enzyme free of any excipient.

According to the invention, the contents of the reactor are maintained under stirring during the depolymerization step. The stirring speed is regulated by the skilled person so as to be sufficient to allow suspension of the plastic/polyester material engaged in the reactor, homogeneity of the temperature and precision of the pH regulation. In particular, stirring is maintained at a speed comprised between 50 and 500 rpm, for example 80 rpm, 100 rpm, 150 rpm, 200 rpm, 250 rpm, 300 rpm, 350 rpm, 400 rpm, 450 rpm, 500 rpm. In a particular embodiment, for a reactor with a volume greater than 1000 L, the stirring is greater than or equal to 300 rpm. In a particular embodiment, for a reactor with a volume greater than 1000 L, the stirring is greater than or equal to 100 rpm.

The depolymerization of the polyester of interest produces acidic monomers that may cause a decrease in the pH of the reactor contents. A base addition can be used to neutralize the acid produced and regulate the pH. In the case of the present invention, the pH can in particular be regulated by the addition of any bases known to the person skilled in the art. In particular, the pH is regulated by the addition of a base selected from sodium hydroxide (NaOH), potassium hydroxide (KOH) and/or ammonia (NH₄OH). In the case of pH regulation by the addition of base, the TA produced will thus associate with the base(s) used so as to form TA salts whose solubility is increased with respect to the TA. Advantageously, the pH is regulated during the depolymerization step by the addition to the reaction medium of a basic solution concentrated to at least 10%±1%, by weight of base based on the total weight of the basic solution (essentially comprising the base and water). Preferentially, the basic solution is concentrated to at least 15%±1% and at most 50%±1%, more preferentially at least 20%±1%. In a particular case, the basic solution is concentrated between 20% and 50%±1%, more preferentially between 20% and 30%±1%, even more preferentially between 20% and 25%±1%. Preferentially, the base is selected from sodium hydroxide (NaOH) and potassium hydroxide (KOH) and the basic solution is concentrated to at least 15% and at most 50%.

The pH is thus regulated to be maintained between 6.5 and 9, so that the terephthalic acid produced is predominantly in the form of solubilized TA salts and/or so as to be at the optimum pH of the enzyme. In a particular embodiment, the pH is regulated between 6.5 and 8.5 during the depolymerization step, preferentially between 7 and 8. In another particular case, the pH is regulated between 7.5 and 8.5. Preferentially the pH is regulated to 8±0.2.

Advantageously, the temperature within the reactor, and thus in the reaction medium, is regulated between 35° C. and 90° C. during the depolymerization step, preferentially between 45° C. and 80° C. In a preferred embodiment of the invention, the temperature is regulated between 55° C. and 80° C., more preferentially between 60° C. and 80° C. In a particular embodiment, the temperature is regulated between 60° C. and 66° C.

In a particular case, the polyester of interest has a glass transition temperature (Tg) greater than 30° C. and the temperature within the reactor is regulated to a temperature less than or equal to the Tg of the polyester of interest. Alternatively or additionally, the temperature is regulated to the optimum temperature of the enzyme used. In a particular embodiment, the polyester of interest is PET with a Tg of about 70° C.±5° C., and the temperature within the reactor is maintained at 60° C.±5° C.

According to the invention, the depolymerization step is conducted for a reaction time of at most 150 h. The reaction time depends, among other things, on the polyester of interest/depolymerization enzyme pair and the desired depolymerization rate of the polyester. The person skilled in the art will know how to adapt the reaction time of the depolymerization step as a function of the above-mentioned criteria. Advantageously, the depolymerization step lasts between 1 h and 120 h, between 1 h and 100 h, between 1 h and 72 h, between 1 h and 48 h, between 1 h and 36 h, between 1 h and 24 h, between 1 h and 12 h, between 1 h and 10 h, between 1 h and 6 h. In a preferred embodiment, the time of the depolymerization step is less than 24 h.

In a preferred embodiment, the above reaction time achieves a depolymerization of the polyester of interest of at least 80%, preferentially at least 85%, 90%, 95%. Preferentially, the depolymerization is conducted down to the monomers, i.e., 80% depolymerization leads to 80% production of monomers (and no or almost no oligomers).

In a particular embodiment, the process for producing TA is carried out from plastic materials comprising PET and an enzyme whose amino acid sequence comprises at least SEQ ID NO: 1, said process allowing depolymerization of at least 80% of the PET in a time of less than 72 h, preferentially depolymerization of at least 90% of the PET is obtained in a time of less than 72 h. In another preferred embodiment, said process allows a depolymerization of at least 80% of the PET in a time shorter than 48 h.

According to the invention, the depolymerization step can be carried out in any reactor usually used in the chemical industry or in biological production, such as a fermenter. Generally speaking, according to the invention, the depolymerization step can be carried out in any tank or reactor whose temperature and pH can be regulated and provided with stirring means to homogenize the medium.

Production of Terephthalic Acid

The process according to the invention makes it possible to produce high concentrations of terephthalic acid in reaction times perfectly compatible with industrial constraints.

More particularly, the process according to the invention makes it possible to obtain at the conclusion of the depolymerization step a terephthalic acid concentration of at least 40 kg/t based on the total weight of the liquid phase of the final reaction medium. Advantageously, the depolymerization step is considered to have been completed when the depolymerization rate of the polyester of interest reaches at least 80%, preferentially at least 90%. In a particular embodiment, the depolymerization step can be stopped by the person skilled in the art when the yields reached are compatible with industrial constraints, i.e., when the depolymerization rate of the polyester of interest reaches 80%±10%, preferentially 90%±5%. Thus, in the context of the invention, the end of the depolymerization step corresponds to the moment when the depolymerization of the polyester is stopped, and/or to the moment when the depolymerization rate of the polyester of interest reaches at least 80%, preferentially at least 90%, and/or to the moment when the step of recovering the TA salts begins.

Preferentially, the concentration of terephthalic acid obtained from the polyester of interest after the depolymerization step is greater than 50 kg/t, 60 kg/t, 70 kg/t, 80 kg/t, 90 kg/t, 100 kg/t, 110 kg/t, 120 kg/t based on the total weight of the liquid phase of the final reaction medium. In a preferred case, the concentration of terephthalic acid obtained from the polyester of interest after the depolymerization step is comprised between 100 kg/t and 115 kg/t±10%.

In a particular case, the concentration of total terephthalic acid (soluble and non-soluble) obtained from the polyester of interest at the conclusion of the depolymerization step is greater than 50 kg/t, 60 kg/t, 70 kg/t, 80 kg/t, 90 kg/t, 100 kg/t, 110 kg/t, 120 kg/t, 130 kg/t, 140 kg/t, 150 kg/t based on the total weight of the final reaction medium (liquid phase and solid phase)

In another particular case, the concentration of total terephthalic acid (soluble and non-soluble) obtained from the polyester of interest at the conclusion of the depolymerization step is greater than 50 kg/t, 60 kg/t, 70 kg/t, 80 kg/t, 90 kg/t, 100 kg/t, 110 kg/t, 120 kg/t, 130 kg/t, 140 kg/t, 150 kg/t based on the total weight of the liquid phase of the final reaction medium+non-soluble TA.

Advantageously, according to the invention, at least 80% by weight of the TA salts produced during the depolymerization step are in solubilized form, preferentially at least 85%, 90%, 95%.

In a particular case, the amount of polyester of interest engaged in the reactor is greater than or equal to 15% by weight based on the total weight of the initial reaction medium, the pH is regulated between 7 and 8 and the temperature at 60° C.±5° C. during the depolymerization step. The concentration of TA in the liquid phase of the final reaction medium after 24 h is advantageously higher than 54 kg/t. In another particular case, the amount of polyester of interest engaged in the reactor is greater than or equal to 15% by weight based on the total weight of the initial reaction medium, the pH is regulated between 7.5 and 8.5 and the temperature at 60° C.±5° C. during the depolymerization step, and the concentration of TA in the liquid phase of the final reaction medium after 24 h is advantageously greater than 77 kg/t, and after 48 h advantageously greater than 84 kg/t.

In another particular case, the amount of polyester of interest engaged in the reactor is greater than or equal to 20% by weight based on the total weight of the initial reaction medium, the pH is regulated between 7 and 8 and the temperature at 60° C.±5° C. during the depolymerization step. The concentration of TA in the liquid phase of the final reaction medium is advantageously greater than 90 kg/t after 24 h. In another particular case, the amount of polyester of interest engaged in the reactor is greater than or equal to 20% by weight based on the total weight of the initial reaction medium, the pH is regulated between 7.5 and 8.5 and the temperature at 60° C.±5° C. during the depolymerization step. The concentration of TA in the liquid phase of the final reaction medium is advantageously greater than 95 kg/t after 24 h and advantageously greater than 100 kg/t after 48 h.

In another particular case, the amount of polyester of interest engaged in the reactor is greater than or equal to 20% by weight based on the total weight of the initial reaction medium, the pH is regulated between 7 and 8, and the temperature between 50° C. and 60° C. during the depolymerization step. The concentration of TA in the liquid phase of the corresponding final reaction medium after 48 h is advantageously greater than 90 kg/t. In a preferred case, the temperature is regulated at 60° C. and the concentration of TA in the liquid phase of the final reaction medium after 48 h is greater than 100 kg/t.

In another particular case, the amount of polyester of interest used in the reactor is greater than or equal to 20% by weight based on the total weight of the initial reaction medium, the pH is regulated between 7 and 8, and the temperature at 60° C. during the depolymerization step, the reactor used has a volume greater than 150 L and the concentration of TA in the liquid phase of the final reaction medium after 48 h is greater than 75 kg/t.

In another particular case, the amount of polyester of interest used in the reactor is greater than or equal to 25% by weight based on the total weight of the initial reaction medium, the pH is regulated between 7 and 8, and the temperature at 60° C. during the depolymerization step, the reactor used has a volume greater than 1000 L and the concentration of TA in the liquid phase of the final reaction medium after 48 h is greater than 120 kg/t.

In another particular case, the process for producing terephthalic acid (TA) according to the invention from at least one PET comprises a PET depolymerization step lasting less than 48 h according to which a plastic material containing PET is brought into contact with at least one cutinase capable of depolymerizing said PET in a stirred reactor, and a step of recovering TA salts in solubilized form, according to which the reactor contains, at the beginning of the depolymerization step, an amount of engaged PET greater than or equal to 20% by weight based on the total weight of the initial reaction medium, the pH is regulated between 7 and 8.5 and the temperature between 60° C. and 80° C. during the depolymerization step, and the concentration of TA in the reactor at the conclusion of the depolymerization step is greater than 100 kg/tin the liquid phase of the final reaction medium.

In another particular case, the process for producing terephthalic acid (TA) according to the invention from at least one plastic material containing PET comprises a PET depolymerization step lasting less than 48 h according to which a plastic material containing PET is brought into contact with at least one cutinase capable of depolymerizing said PET in a stirred reactor, and a step of recovering TA salts in solubilized form, according to which the reactor contains, at the beginning of the depolymerization step, an amount of engaged PET comprised between 15% and 25% by weight based on the total weight of the initial reaction medium, the pH is regulated between 7.5 and 8.5 and the temperature between 60° C. and 80° C. during the depolymerization step, and the concentration of TA in the liquid phase of the final reaction medium in the reactor at the conclusion of the depolymerization step is greater than 100 kg/t.

In another particular case, the amount of polyester of interest engaged in the reactor is comprised between 15% and 25% by weight based on the total weight of the initial reaction medium, the basic solution used is concentrated to 15%±1%, and the concentration of TA in the liquid phase of the final reaction medium after 48 h is greater than 80 kg/t, preferentially greater than 100 kg/t. Preferentially the amount of polyester of interest engaged in the reactor is greater than 20%±2% by weight based on the total weight of the initial reaction medium, and the concentration of TA in the liquid phase of the final reaction medium after 48 h is greater than 110 kg/t. Advantageously the pH is regulated between 7.5 and 8.5.

In another particular case, the amount of polyester of interest engaged in the reactor is comprised between 15% and 25% by weight based on the total weight of the initial reaction medium, the basic solution used is concentrated to 20%±1%, and the concentration of TA in the liquid phase of the final reaction medium after 48 h is greater than 85 kg/t, preferentially greater than 110 kg/t. Preferentially the amount of polyester of interest engaged in the reactor is greater than 20%±2%, and the concentration of TA in the liquid phase of the final reaction medium after 48 h is greater than 110 kg/t. Advantageously the pH is regulated between 7.5 and 8.5.

In another particular case, the amount of polyester of interest engaged in the reactor is comprised between 15% and 25% by weight based on the total weight of the initial reaction medium, the basic solution used is concentrated to 25%±1%, and the concentration of TA in the liquid phase of the final reaction medium after 48 h is greater than 90 kg/t, preferentially greater than 110 kg/t. Preferentially the amount of polyester of interest engaged in the reactor is greater than 20%±2%, and the concentration of TA in the liquid phase of the final reaction medium after 48 h is greater than 110 kg/t. Advantageously the pH is regulated between 7.5 and 8.5. In another particular case, the plastic material is selected from fibers and/or waste fibers and/or textiles, and the amount of polyester of interest engaged in the reactor is comprised between 15% and 25% by weight based on the total weight of the initial reaction medium, the basic solution used is concentrated to 20%±1%, and the concentration of TA in the liquid phase of the final reaction medium after 48 h is greater than 80 kg/t, preferentially greater than 90 kg/t. Advantageously the pH is regulated between 7.5 and 8.5.

According to the invention, it is possible to easily recover the solubilized terephthalate salts (and/or terephthalic acid) in the liquid phase of the final reaction medium from the reactor content. In a particular embodiment, the step of recovering the solubilized TA salts comprises, in particular, a separation of the liquid phase containing the TA salts from the rest of the reaction medium. In a particular case, this step of separating the solubilized terephthalate salts in the liquid phase is carried out by filtration of the reaction medium allowing recovery, in a solution, of the terephthalate salts in solubilized form. The filtration cut-off can be adapted by the person skilled in the art. The separation step can also be carried out by centrifugation or any other technique known to the skilled person.

The separation residue (“retentate”, i.e., the solid phase comprising in particular the residual non-degraded polyester and/or the other polymers contained in the plastic material and/or non-solubilized TA and TA salts) can be recycled into the reactor in order to undergo a new depolymerization step. It can also be washed with water in order to allow the dissolution of the non-solubilized TA salts in the liquid phase and thus allow their recovery in solubilized form in the wash water.

Advantageously, the recovered TA salts can be reused in the form of TA, particularly for the production of new polyesters. According to the invention, the process comprises an additional step of TA recovery by precipitation of the TA contained in said salts.

In a particular embodiment, this precipitation of the TA is achieved by acidification of the medium. For example, the filtered solution (i.e., the liquid phase of the final reaction medium) containing the solubilized terephthalate salt(s) may be subjected to some or all of the following steps (this sequence of steps also being suitable for the above-mentioned wash water):
1. Purification of the filtered solution (and/or wash water) by subjecting the solution to one or more steps selected from ultrafiltration, decolorization on carbon, passage over ion exchangers and chromatography; and/or
2. Precipitation of the terephthalic acid contained in the filtered solution, wash water or purified solution by acidifying the solution with a mineral acid (which may for example be selected from the following acids: sulfuric acid, hydrochloric acid, phosphoric acid, or nitric acid) or with an organic acid (of the acetic acid type), alone or in mixture. The solution can also be acidified by CO₂ overpressure. This step also induces a solubilization of the salts produced at the same time as the precipitation of the TA; and/or
3. Filtration of the solution containing precipitated terephthalic acid to recover terephthalic acid in solid form; and/or
4. Washing (preferentially several successive washes) of the terephthalic acid with purified water and drying to obtain purified TA (“CTA”).

The CTA obtained can then be crystallized and optionally further purified to obtain purified and crystallized TA (“PTA”) by any techniques known to the skilled person.

The CTA and/or PTA resulting from the process of the invention can be reused alone or as a mixture. In particular, they can be repolymerized, alone or as a mixture, for the synthesis of a polyester containing at least one terephthalic acid unit, identical to or different from the polyester of interest engaged in the reactor.

The salts and other monomer(s) obtained in the filtrate from step 3 can be extracted and purified by techniques known to the skilled person in order to be reused and/or recovered.

In another embodiment, the filtered solution (i.e., the liquid phase of the final reaction medium) containing the solubilized terephthalate salts is subjected to a concentration step that can be carried out by any method allowing the removal of the water contained in the solution (e.g., evaporation) and thus leading to the precipitation of the terephthalate salts in solid form. The TA salts in solid form are recovered by filtration and then put back into solution before acidification of the solution by an acid (mineral or organic) to precipitate terephthalic acid. This concentration step can be carried out at any time during the purification process and will be followed by a step of acidification of the medium. The above-mentioned step 4 can then be carried out in order to obtain CTA.

According to the invention, it is possible to recover at least one other monomer from the depolymerization of the polyester of interest. The process according to the invention thus comprises an additional step according to which at least one other monomer constituting the polyester of interest is recovered. In an embodiment, the polyester of interest is PET, and ethylene glycol monomers are recovered at the conclusion of the depolymerization step in addition to terephthalic acid. In another embodiment, the polyester of interest is PTT, and propanediol (or propylene glycol) monomers are recovered from the depolymerization step in addition to terephthalic acid. In another embodiment, the polyester of interest is PBT, and butanediol monomers in addition to terephthalic acid are recovered from the depolymerization step. In another embodiment, the polyester of interest is PBAT, and butanediol and/or adipic acid monomers in addition to terephthalic acid are recovered from the depolymerization step. In another embodiment, the polyester of interest is PCT, and cyclohexanedimethanol monomers in addition to terephthalic acid are recovered from the depolymerization step. In another embodiment, the polyester of interest is PEIT, and ethylene glycol and/or isosorbide monomers are recovered from the depolymerization step in addition to terephthalic acid.

According to the invention, in addition to terephthalic acid, it is also possible to recover oligomers, i.e., molecules comprising between 2 and 20 monomers, including at least one terephthalic acid unit. In a particular case, the polyester of interest is PET and oligomers such as methyl-2-hydroxyethyl terephthalate (MHET), bis(2-hydroxyethyl) terephthalate (BHET), and dimethyl terephthalate (DMT) are recovered at the conclusion of the depolymerization step in addition to terephthalic acid.

The invention also relates to the use of a reactor having a volume greater than 1 L for the implementation of a process for producing terephthalic acid (TA) which comprises a step of depolymerization of a polyester of interest according to which said polyester is brought into contact with at least one enzyme capable of depolymerizing said polyester and carried out in said reactor, and a step of recovering the TA salts in solubilized form.

Preferentially, the object of the invention is to use a reactor having a volume greater than 2 L, 5 L, 10 L, 100 L, 1000 L, 10 000 L, 100 000 L, 400 000 L for the implementation of a process for producing terephthalic acid (TA) described above.

The invention also has as its object a reactor with a volume of at least 1000 liters containing at least one polyester of interest comprising at least one TA unit and at least one enzyme capable of depolymerizing said polyester of interest, and in which at least one step of enzymatic depolymerization of said polyester of interest is carried out, the amount of polyester engaged in the reactor being greater than 10% by weight based on the total weight of the initial reaction medium, and the concentration of TA in the liquid phase of the final reaction medium being greater than 40 kg/t. Advantageously, the amount of polyester engaged in the reactor is comprised between 15% and 25% by weight based on the total weight of the initial reaction medium, the concentration of TA in the liquid phase of the final reaction medium is greater than 100 kg/t, and the pH is regulated during the depolymerization step between 7.5 and 8.5 by the addition of a basic solution concentrated between 15% and 50% by weight of base based on the total weight of the basic solution, preferentially by the addition of a basic solution concentrated between 15% and 25%.

According to the invention, the process is implemented in a discontinuous manner, in the form of a batch treatment. Generally, the process thus comprises a depolymerization step carried out for a given time from a given volume of initial reaction medium, followed by a step of recovering the TA salts produced. At the end of the depolymerization step, the reactor can be drained so as to recover the whole reaction medium, which can then undergo the various steps described above so as to separate the solubilized terephthalate salts from the rest of the reaction medium, purify them and recover the TA.

All the features of the process for producing terephthalic acid according to the invention described above can also be applied to a process for producing monomers close to terephthalic acid, and particularly to a process for producing 2,5-furandicarboxylic acid (FDCA) from polyethylene furanoate (PEF).

### EXAMPLES

### Example 1: Production of Terephthalic Acid in a Reactor Comprising an Amount of Engaged PET Greater than 10% by Weight Based on the Total Weight of the Initial Reaction Medium

For this experimental design, terephthalic acid production was performed in flat-bottom stirred reactors with a total volume of 500 mL (MiniBioreactors, Global Process Concept). Each reactor was equipped with a temperature probe and a pH probe (Hamilton, EasyFerm HB BioArc 120). The regulation of these two parameters at the set values was ensured by internal PID controllers in the C-bio software (Global Process Concept). A 3 cm diameter marine paddle attached to the central shaft rotating at 300, 400 or 450 rpm provided the stirring of the reaction medium. Several basic solutions were used for pH regulation: either 6 M NaOH (i.e., concentrated to 19.4%), or 6 M NH₄OH (concentrated to 17.4%), or 6 M KOH (concentrated to 25%).

For experiments A to C, terephthalic acid (TA) production was performed from colorless bottle preforms, composed of 100% amorphous PET, reduced to fine powder by cryo-grinding (D50=850 μm).

For experiments D to K, terephthalic acid production was carried out using colored and washed plastic flakes from the PET waste recycling stream, which were kindly donated to us. These plastic materials, composed of 98% m/m PET, underwent an extrusion step, followed by a rapid cooling allowing the amorphization of the PET contained in the waste. The extruder used for amorphization was a Leistritz ZSE 18 MAXX twin screw extruder. The temperature of the heating zones was set to 260° C. on the first 4 zones and 250° C. on the last 6 zones and a screw rotation speed of 200 rpm. The rod arriving at the extruder head is then immediately immersed in a water bath at 10° C. The degree of crystallinity of the PET after this amorphization step was evaluated at about 19% (by DSC). The resulting rod was granulated and then reduced to a fine powder using a micronizer (1 mm grid). The powder was then subjected to a 500 μm sieve to recover only the powders smaller than this size.

The enzyme used was LC-Cutinase, an enzyme known to depolymerize PET (SEQ ID NO: 1, corresponding to amino acids 36 to 293 of the sequence described in Sulaiman et al., Appl Environ Microbiol. 2012 March). It was produced by fermentation of a recombinant microorganism in liquid medium. The PET-degrading enzyme was added at a weight ratio of 1:1000 or 2:1000 per amount of engaged PET.

These plastic materials or waste plastics were fed into the reactor so that the amount of engaged PET at the beginning of the depolymerization step was comprised between 5% and 40% based on the total weight of the initial reaction medium. Phosphate buffer is added to the plastic materials and enzyme to reach the total weight of the initial reaction medium.

All the parameters associated with the depolymerization step of the different processes tested are shown in Tables 1A and 1B below:

TABLE 1A
Parameters used during the processes A-C
for producing TA from bottle preforms

| Tests | A | B | C |
|---|---|---|---|
| Plastic materials used comprising the | Bottle Preform - 100% PET - | | |
| polyester of interest | Micronized (<1 mm) | | |
| PET engaged to total weight of initial | 5.0% | 15.0% | 20.0% |
| reaction medium | | | |
| Total weight of the initial reaction | 237 | 265 | 281 |
| medium (g) | | | |

| | |
|---|---|
| Enzyme/PET weight ratio | 1:1000 |
| Liquid of the reaction medium | 10 mM phosphate buffer pH 7 |
| Temperature during the depolymerization | 60° C. |
| step | |
| Stirring (rpm) during the depolymerization | 300 |
| step | |
| pH regulation setpoint during the | 7.00 |
| depolymerization step | |
| Basic solution used for pH regulation | 6M NH₄OH (17.4%) |

TABLE 1B
Parameters used during processes D-K for producing TA from waste plastics

| Tests | D | E | F | G | H | I | J | K |
|---|---|---|---|---|---|---|---|---|
| Plastic materials used | Washed colored flakes - 98% PET Amorphous -Micronized | | | | | | | |
| comprising the polyester of | (<500 μm) | | | | | | | |
| interest | | | | | | | | |
| PET engaged to total | 10.0% | 20.0% | 25.0% | 30.0% | 40.0% | 20.0% | 30.0% | 40.0% |
| weight of initial reaction | | | | | | | | |
| medium | | | | | | | | |
| Total weight of the initial | 282 | 281 | 281 | 282 | 240 | 281 | 281 | 280 |
| reaction medium (g) | | | | | | | | |

| Enzyme/PET weight ratio | 2:1000 | | | | |
|---|---|---|---|---|---|
| Liquid of the reaction | 100 mM Phosphate Buffer pH 8 | | | | |
| medium | | | | | |
| Temperature during the | 60° C. | | | | |
| depolymerization step | | | | | |
| Stirring (rpm) during the | 300 | 400 | 300 | 450 | 450 |
| depolymerization step | | | | | |

| pH regulation setpoint | 8.00 | |
|---|---|---|
| during the | | |
| depolymerization step | | |
| Basic solution used for pH | 19.4% NaOH (6M) | 25% KOH (6M) |
| regulation | | |

Regular sampling was used to monitor the kinetics of terephthalic acid production. The solid phase (including undegraded plastic materials) was first separated from the liquid phase, containing terephthalate salts in soluble form, by centrifugation (D100-DragonLab).

The concentration of TA was determined by chromatography (UHPLC). For this purpose, 1 mL of methanol and 100 μL of 6 M HCl were added to the diluted sample to decomplex the TA salts. If necessary, dilutions were made on the samples with RO water. The prepared sample was filtered on a 0.22 μM cellulose filter and 20 μL was injected on the chromatographic column. The HPLC system used was the model 3000 UHPLC system (Thermo Fisher Scientific, Inc. Waltham, Mass., USA), including a pump, an automatic sampling system, a column thermostated at 25° C. and a UV detector at 240 nm. Three eluents were used: 10 mM H₂SO₄ (eluent A); ultrapure water (eluent B) and methanol (eluent C). Terephthalic acid is separated from the other molecules by a gradient between these three solvents. Terephthalic acid is measured according to standard standards prepared from commercial terephthalic acid (Acros Organics).

The concentration of terephthalic acid produced after 24 h for the different tests is shown in Tables 2A and 2B below.

TABLE 2A
Concentration and soluble fraction of terephthalic acid obtained after
the depolymerization steps with parameters described in Table 1A.

| Tests | A | B | C |
|---|---|---|---|
| Plastic materials used comprising | Bottle Preforms - 100% PET - | | |
| the polyester of interest | Micronizec (<1 mm) | | |
| PET engaged to total weight of | 5.0% | 15.0% | 20.0% |
| initial reaction medium | | | |
| TA kg/t based on the total weight | 14.4 | 54.2 | 68.7 |
| of the liquid phase of the | | | |
| final reaction medium - 24 h | | | |
| Fraction of Soluble TA salts | 100% | 100% | 100% |

The process for producing terephthalic acid according to the invention thus makes it possible to reach after 24 h a TA concentration greater than 54 kg/t and 69 kg/t, in reactors containing at the beginning of the depolymerization step an amount of engaged polyester equal to 15% and 20% by weight, respectively, based on the total weight of the initial reaction medium, the pH being regulated at 7 during the depolymerization step.

TABLE 2B
Concentration and soluble fraction of terephthalic acid obtained after
the depolymerization steps with the parameters described in Table 1B

| Tests | D | E | F | G | H | I | J | K |
|---|---|---|---|---|---|---|---|---|
| Plastic materials used | Washed colored flakes - 98% PET Amorphous -Micronized | | | | | | | |
| comprising the polyester of | (<500 μm) | | | | | | | |
| interest | | | | | | | | |
| PET engaged to total weight | 10.0% | 20.0% | 25.0% | 30.0% | 40.0% | 20.0% | 30.0% | 40.0% |
| of initial reaction medium | | | | | | | | |
| Total TA (soluble and non- | 56.9 | 95.8 | 106.1 | 109.8 | 131.5 | 77.2 | 94.9 | 118.5 |
| soluble) kg/t of [liquid | | | | | | | | |
| phase of final reaction | | | | | | | | |
| medium + non-soluble | | | | | | | | |
| TA]- 24 h | | | | | | | | |
| TA kg/t of liquid phase of | 56.9 | 95.8 | 106.1 | 109.8 | 119.7 | 77.2 | 94.9 | 118.5 |
| final reaction medium - | | | | | | | | |
| 24 h | | | | | | | | |
| Fraction of Soluble TA salts | 100% | 100% | 100% | 100% | 91% | 100% | 100% | 100% |

The process for producing terephthalic acid according to the invention thus makes it possible to reach after 24 h a TA concentration greater than 57 kg/t, 77 kg/t, 106 kg/t, 95 kg/t and 118 kg/t in reactors containing at the beginning of the depolymerization step an amount of engaged polyester respectively equal to 10%, 20%, 25%, 30% and 40% by weight based on the total weight of the initial reaction medium, the pH being regulated at 8 during the depolymerization step.

### Example 2: Production of Terephthalic Acid in a Reactor Comprising an Amount of Engaged PET Equal to 20% by Weight Based on the Total Weight of the Initial Reaction Medium, the pH and the Temperature being Regulated, During the Depolymerization Step, at Values Fixed Between 7 and 8, and Between 40° C. and 60° C., Respectively

For this second experimental design, all the tests were carried out in the same stirred reactors as described in Example 1 with stirring during the depolymerization step at 300 rpm. The basic solution used for pH regulation was 19.4% NaOH (6 M).

The production of terephthalic acid (TA) was carried out using colored and washed plastic flakes from the PET waste recycling stream identical to those used for experiments D to H in Example 1, except for the final sieving. The resulting powders therefore have a particle size of less than 1 mm. The PET portion of this waste plastic constituted 20% of the mass engaged at the beginning of the PET depolymerization step based on the total weight of the initial reaction medium.

The enzyme used is identical to that used in Example 1. It was added at a weight ratio of 2:1000 per amount of PET used.

Phosphate buffer (100 mM, pH 8) is added to the plastic materials and enzyme to reach the total weight of the initial reaction medium.

Three set temperatures were tested, as well as three pH values. The main information is summarized in the following Table 3:

TABLE 3
Parameters used during the different processes for the production
of TA from waste plastics introduced at 20% based on the
total weight of the initial reaction medium.

| | | | | | |
|---|---|---|---|---|---|
| Tests | A | B | C | D | E |

| Plastic materials used comprising | Washed colored plastic | | | |
|---|---|---|---|---|
| the polyester of interest | flakes - 98% PET Amorphous - | | | |
| | Micronized (<1 mm) | | | |
| PET engaged to total weight of | 20.0% of 281 g | | | |
| initial reaction medium | | | | |
| Temperature (° C.) during the | 60° C. | 50° C. | 40° C. | 60° C. |
| depolymerization step | | | | |

| | | | |
|---|---|---|---|
| pH regulation setpoint during the | 8.0 | 7.0 | 7.5 |
| depolymerization step | | | |

Regular sampling was used to monitor the kinetics of terephthalic acid production and the TA concentration was determined in a similar manner to Example 1.

For the conditions described above, the following results were obtained at 48 h or 72 h.

TABLE 4
Concentration of terephthalic acid obtained from the depolymerization
steps at the beginning of which the amount of engaged PET
is equal to 20% by weight based on the weight of the initial
reaction medium, and whose temperature and pH parameters
were regulated as described in Table 3.

| Tests | A | B | C | D | E |
|---|---|---|---|---|---|
| Temperature (° C.) during | 60° C. | 50° C. | 40° C. | 60° C. | |
| the depolymerization step | | | | | |
| pH regulation setpoint during | 8.0 | | | 7.0 | 7.5 |
| the depolymerization step | | | | | |
| TA kg/t based on the total | 114 | / | / | 104 | 106 |
| weight of the liquid phase | | | | | |
| of the final reaction medium | | | | | |
| at 48 h | | | | | |
| TA kg/t based on the total | 113 | 94 | 41 | / | / |
| weight of the liquid phase of | | | | | |
| the reaction medium at 72 h | | | | | |

The process for producing terephthalic acid according to the invention carried out in a reactor containing, at the beginning of the depolymerization step, an amount of engaged polyester respectively equal to 20%, makes it possible to reach, after 48 h and 72 h, a TA concentration greater than 40 kg/t. At 60° C., this process makes it possible to reach after 48 h a concentration greater than 90 kg/t for a pH between 7 and 8. At 50° C., this process makes it possible to reach after 72 h a concentration greater than 90 kg/t for a pH between 7 and 8.

### Example 3: Production of Terephthalic Acid in a Reactor Comprising an Amount of Engaged PET Equal to 20% by Weight, Contained in a Plastic Material in the Form of Granules

For this third experimental design, all the tests were performed in the same stirred reactors as described in Example 1. The temperature setpoint was fixed at 60° C. and the pH was regulated at 8.0 using 19.4% NaOH (6 M) as a basic solution.

The production of terephthalic acid (TA) was performed using PET Lighter C93 from Resinex. The PET was amorphized by extrusion, followed by rapid cooling. The extruder used for amorphization was a Leistritz ZSE 18 MAXX twin-screw extruder, with heating zones set between 285° C. and 304° C. The degree of crystallinity of the PET obtained in granule form after this amorphization step was estimated to be about 13% (by DSC).

The granules were fed into the reactor at 20% by weight based on the weight of the initial reaction medium. The enzyme used was identical to that used in Example 1. It was added at a weight ratio of 1:1000 of engaged PET. Potassium phosphate buffer at 10 mM pH 8 is added to the plastic materials and enzyme to reach the total weight of the initial reaction medium.

Regular sampling as described in Example 1 was used to monitor the kinetics of terephthalic acid production. The terephthalic acid produced was measured by HPLC according to the protocol described in Example 1. After 88 h, 62 kg/t of TA based on the total weight of the liquid phase of the final reaction medium is obtained. The results indicate that it is also possible to achieve the performance claimed in the present application when the plastic material containing the polyester of interest is introduced in the form of granules.

### Example 4: Terephthalic Acid Production at Different Reactor Scales

In this example, different stirred reactors were used to produce terephthalic acid. These reactors of increasing size are used to validate the scaling up of the process and its use on an industrial or semi-industrial scale.

For these tests, two types of plastic materials were used. In tests A, B, C, E and F it is the washed colored flakes described in Example 1 (98% PET Amorphous, micronized <500 μm), while for Example D it is bottle preforms also described in Example 1 (100% PET, micronized <1 mm). These plastic materials are engaged in such a way as to obtain an amount of engaged PET of 20% by weight based on the weight of the initial reaction medium. The temperature setpoint was set at 60° C. and the pH was regulated to 8.0 or 7.0 using 19.4% NaOH (Tests A to D) or 25% m/m NaOH (Test E and F). For Tests A, B, C, D and E, the enzyme used is identical to that used in Example 1. For Test F, it is a variant of the enzyme of Example 1 (with the following mutations SEQ ID NO: 1+F2081+D203C+S248C+V170I+Y92G) also obtained by fermentation of a recombinant microorganism. They were added at a weight ratio of 1:1000 per weight of PET engaged for tests A to D and 2:1000 for tests E and F.

For Test A, a 500 mL flat-bottom reactor described in Example 1 was used. Stirring was provided by a 3 cm diameter marine paddle attached to the central shaft. The stirring speed was set at 300 rpm. 56.3 g of plastic materials was engaged.

For Test B, a dished-bottom reactor with a total volume of 5 L (Global Process Concept) was used. The reactor was equipped with a temperature probe and a pH probe (Hamilton, EasyFerm HB BioArc 325). The regulation of these two parameters at the set values was ensured by PID controllers internal to the C-bio software (Global Process Concept). A 5.5 cm diameter marine paddle attached to the central shaft rotating at 200 rpm provided the stirring of the reaction medium. 375 g of plastic materials was engaged.

For Test C, a dished-bottom reactor with a total volume of 40 L was used. The reactor was equipped with a temperature probe and a pH probe (Rosemount analytical HX338-05). A 14 cm diameter marine paddle attached to the central shaft rotating at 150 rpm was used to stir the reaction medium. 4 kg of plastic materials were used.

For Test D, a dished-bottom reactor with a total volume of 150 L was used. The reactor was equipped with a temperature probe and a pH probe (EasyFerm BioArc 120, Hamilton). A 25 cm diameter marine paddle attached to the central shaft rotating at 80 rpm was used to stir the reaction medium. 14 kg of plastic materials was used.

For Tests E and F, a flat-bottom reactor with a total volume of 1000 L was used. The reactor was equipped with a temperature probe and a pH probe (In Pro3100/SG/325, Mettler Toledo). A marine paddle of variable diameter was used to stir the reaction medium. 75 kg of plastic materials was used.

It is understood that the curved or flat shape of the bottom of the reactor does not affect the process and that the shape of the bottoms is interchangeable.

The main information has been summarized in the following Table 5:

TABLE 5
Parameters used during the different processes for the production of
TA from plastic materials introduced with 20% engaged PET to the total
weight of the initial reaction medium, in 500 mL to 1000 L reactors.

| Tests | A | B | C | D | E | F |
|---|---|---|---|---|---|---|
| Reactor size | 500 mL | 5 L | 40 L | 150 L | 1000 L | |
| Plastic materials | Washed colored flakes - 98% | | | Bottle Preforms - | Washed colored flakes | |
| used comprising the | PET Amorphous - | | | 100% PET - | | |
| polyester of interest | Micronized (<500 μm) | | | Micronized (<1 mm) | | |
| PET engaged to total | 20% | | | | | |
| weight of initial | | | | | | |
| reaction medium | | | | | | |
| Liquid of the | 10 mM | | RO water | RO water | RO water | Water |
| reaction medium | phosphate buffer | | | | | network |
| Stirring (rpm) during | 300 | 200 | 150 | 80 | 100 rpm | 100 rpm |
| the depolymerization | | | | | | |
| step | | | | | | |

| | | | | |
|---|---|---|---|---|
| pH regulation | 8.0 | 7.0 | 8.0 | 8.0 |
| setpoint during the | | | | |
| depolymerization | | | | |
| step | | | | |

Regular sampling as described in Example 1 was used to monitor the kinetics of terephthalic acid production. The TA concentration was determined by UHPLC (described in Example 1).

Thus, for the different reactions, the results obtained are detailed in Table 6:

TABLE 6
Concentration of terephthalic acid obtained from the depolymerization
steps of the processes described in Table 5.

| Tests | A | B | C | D | E | F |
|---|---|---|---|---|---|---|
| Reactor size | 500 mL | 5 L | 40 L | 150 L | 1000 L | 1000 L |
| Time (h) | 16 | 16 | 30 | 48 | 48 | 44 |
| Total TA (soluble | 91 | 90 | 101 | 78 | 122 | 116 |
| and non-soluble) in | | | | | | |
| kg/t of [liquid phase | | | | | | |
| of final reaction | | | | | | |
| medium + non- | | | | | | |
| soluble TA], | | | | | | |
| TA kg/t based on | 91 | 90 | 101 | 78 | 115.9 | 113.7 |
| the total weight of | | | | | | |
| the liquid phase of | | | | | | |
| the final reaction | | | | | | |
| medium | | | | | | |
| Fraction of Soluble | 100% | 100% | 100% | 100% | 95% | 98% |
| TA salts | | | | | | |

Thus, concentrations higher than 78 kg/t of terephthalic acid based on the total weight of the liquid phase of the final reaction medium are obtained under each of the described conditions. In particular, concentrations above 110 kg/t of terephthalic acid based on the total weight of the liquid phase of the final reaction medium are obtained in 1000 L reactors.

### Example 5: Production of Terephthalic Acid in a Reactor Containing PET from Textile Waste

For these experiments, terephthalic acid production was performed in dished-bottom reactors with a total volume of 5 L (Global Process Concept) (described in Example 4).

For Experiment A, terephthalic acid production was carried out from used, shredded clothing textiles without metals and hard points (buttons, zippers, etc.). The shredded textile pieces have a size of 5×5 cm and contain about 83% PET.

For Experiment B, TA was produced from production scrap from a waterj et weaving process, where the material is in the form of continuous thread clusters and contains roughly 100% PET.

These textile materials underwent a drying step at 60° C. for 16 hours and then an extrusion step, followed by a rapid cooling allowing the amorphization of the PET contained in the waste. The same extruder as for Example 1 was used. The temperatures of the heating zones were adjusted according to the following profile:
265° C.-265° C.-265° C.-255° C.-255° C.-250° C.-250° C.-245° C.-245° C.-245° C.

The screw speed was set to 150 rpm. The introduction of the material into the extruder was done manually. The cooling step and the powder reduction step were identical to those used in Example 1. The degree of crystallinity of the samples was estimated to be about 18% for the sample in Experiment A and is less than 10% for the sample in Experiment B.

The enzyme used is identical to that used in Example 4 for Test F. These plastic products or waste plastics were fed into the reactor so that the amount of engaged PET at the beginning of the depolymerization step is comprised between 16.6% and 20% based on the total weight of the initial reaction medium. Phosphate buffer is added to the plastic materials and enzyme to reach the total weight of the initial reaction medium.

The set of parameters associated with the depolymerization step of Experiments A and B is shown in Table 7

TABLE 7
Parameters used during Experiments A and
B of TA production from textile waste.

| Tests | A | B |
|---|---|---|
| Plastic materials used | Amorphous -micronized | Amorphous -micronized |
| comprising the polyester of | (<500 μm) shredded used | (<500 μm) weaving waste - |
| interest | clothing - 83% PET | 100% PET |
| PET engaged to total weight | 16.6% | 20.00% |
| of initial reaction medium | | |
| Total weight of the initial | 2600 | 2600 |
| reaction medium (g) | | |
| Enzyme/PET weight ratio | 2:1000 | 2:1000 |
| Liquid of the reaction medium | 100 mM Phosphate | 100 mM Phosphate |
| | Buffer pH 8 | Buffer pH 8 |
| Temperature during the | 60° C. | 60° C. |
| depolymerization step | | |
| Stirring (rpm) during the | 300 | 300 |
| depolymerization step | | |
| pH regulation setpoint during | 8.0 | 8.0 |
| the depolymerization step | | |
| Basic solution used for pH | 19.4% NaOH | 19.4% NaOH |
| regulation | | |

Regular sampling as described in Example 1 was used to monitor the kinetics of terephthalic acid production. The terephthalic acid produced was measured by HPLC according to the protocol described in Example 1

The concentrations of terephthalic acid produced after 24 h and 48 h for the different tests are shown in Table 8 below.

TABLE 8
Concentration of terephthalic acid obtained at the conclusion of the
depolymerization steps whose parameters are described in Table 7.

| Tests | A | B |
|---|---|---|
| TA kg/t based on the total weight of the | 77 | 95 |
| liquid phase of the final reaction | | |
| medium - 24 h | | |
| TA kg/t based on the total weight of the | 84 | 102 |
| liquid phase of the final reaction | | |
| medium - 48 h | | |

Thus, concentrations higher than 75 kg/t of terephthalic acid based on the total weight of the liquid phase of the final reaction medium are obtained in each of the conditions described.

1-15. (canceled)

16. A process for producing terephthalic acid (TA) from at least one polyester of interest comprising at least one TA unit, comprising a step of enzymatic depolymerization of the polyester, according to which said polyester is brought into contact with at least one enzyme capable of depolymerizing said polyester in a stirred reactor, and a step of recovering TA salts in solubilized form wherein the amount of polyester engaged in the reactor is greater than or equal to 11% by weight based on the total weight of the initial reaction medium, wherein the pH is regulated between 6.5 and 9 during the depolymerization step, and wherein the concentration of TA in the liquid phase of the final reaction medium is greater than 40 kg/t.

17. The process as claimed in claim 16, wherein the ratio by weight of the amount of enzyme engaged to the amount of polyester of interest engaged is comprised between 0.01:1000 and 3:1000.

18. The process as claimed in claim 16, wherein the polyester of interest is selected from polyethylene terephthalate (PET), polyethylene terephthalate glycol (PETG), polyethylene co-isosorbide-terephthalate (PEIT), polytrimethylene terephthalate (PTT), polybutylene adipate terephthalate (PBAT), polycyclohexylenedimethylene terephthalate (PCT) and polybutylene terephthalate (PBT).

19. The process as claimed in claim 16, wherein the amount of polyester of interest engaged in the reactor is comprised between 11% and 20% by weight based on the total weight of the initial reaction medium.

20. The process as claimed in claim 16, wherein the amount of polyester of interest engaged in the reactor is equal to 15%, +/−2% by weight based on the total weight of the initial reaction medium.

21. The process as claimed in claim 16, wherein the amount of polyester of interest engaged in the reactor is greater than or equal to 15% by weight based on the total weight of the initial reaction medium.

22. The process as claimed in claim 16, wherein the concentration of TA in the liquid phase of the final reaction medium is greater than 50 kg/t, 60 kg/t, 70 kg/t, 80 kg/t, 100 kg/t or 120 kg/t.

23. The process as claimed in claim 16, wherein the polyester of interest is in powder form.

24. The process as claimed in claim 16, wherein during the depolymerization step the pH is regulated between 6.5 and 8.5.

25. The process as claimed in claim 16, wherein the depolymerization step lasts at most 150 h.

26. The process as claimed in claim 16, wherein the step of recovering the solubilized TA salts comprises a step of separating the liquid phase containing the TA salts from the remaining reaction medium.

27. The process as claimed in claim 16, wherein the process comprises an additional step of recovering the TA by precipitating the TA contained in the TA salts.

28. The process as claimed in claim 27, wherein the process comprises further to the step of separating the TA salts, a step of recovering the TA by precipitation.

29. The process as claimed in claim 28, wherein precipitation of TA is achieved by acidification of the medium.

30. The process as claimed in claim 29, wherein the liquid phase containing the TA salts obtained from the step of separating is subjected to a concentration step and/or a purification step prior to the precipitation, wherein the purification step is performed by subjecting the liquid phase to one or more steps selected from ultrafiltration, decolorization on carbon, passage over ion exchangers and chromatography.

31. The process as claimed in claim 16, wherein the polyester of interest is PET and the enzyme is a cutinase capable of depolymerizing PET

32. The process as claimed in claim 16, wherein the enzyme is selected from enzymes having an amino acid sequence having at least 75% identity with SEQ ID NO: 1.

33. The process as claimed in claim 16, wherein the pH is regulated during the depolymerization step by the addition to the reaction medium of a basic solution concentrated to at least 10%±1%, by weight of base based on the total weight of the basic solution.

34. The process as claimed in claim 16, wherein the amount of polyester of interest engaged in the reactor is comprised between 15% and 25%, the pH is regulated between 7.5 and 8.5 during the depolymerization step by the addition to the reaction medium of a basic solution concentrated to at least 15%±1%, and wherein the concentration of TA in the liquid phase of the final reaction medium is greater than 100 kg/t.

35. The process as claimed in claim 16, wherein the temperature is regulated between 60° C. and 80° C.

36. The process as claimed in claim 16, wherein the process is carried out in a reactor whose volume is greater than 1000 L.

37. A reactor with a volume of at least 1000 L in which at least one step of enzymatic depolymerization of a polyester of interest comprising at least one TA unit is implemented, wherein the amount of polyester engaged in the reactor is greater than or equal to 11% by weight based on the total weight of the initial reaction medium, and wherein the concentration of TA in the liquid phase of the final reaction medium is greater than 40 kg/t.

38. The reactor as claimed in claim 37, wherein the amount of polyester of interest engaged in the reactor is comprised between 15% and 25%, the pH and the temperature in the reactor are regulated between 7.5 and 8.5 and between 60° C. and 80° C. respectively during the depolymerization step, and the concentration of TA in the liquid phase of the final reaction medium is greater than 100 kg/t.
